(19) United States
(12) Patent Application Publication
(10) Pub. No.: US 2023/0104221 A1
(43) Pub. Date: Apr. 6, 2023

(54) REJUVENATING LASER DRILLING APPARATUS

(71) Applicant: Oren Aharon, Haifa (IL)

(72) Inventor: Oren Aharon, Haifa (IL)

(21) Appl. No.: 17/494,859

(22) Filed: Oct. 6, 2021

(51) Int. Cl. A61B 18/20; A61B 90/00

(52) U.S. Cl. CPC A61B 18/203; A61B 90/361; A61B 2018/00452

(57) ABSTRACT

Rejuvenating laser treatments are well known technologies used for a relatively long time to ameliorate wrinkles and other skin imperfections. A leading technology for this area is the Fraxel Laser, which relies on applying multiple collimated (not focused) laser beams on a given skin surface to produce a micro-column abrasion into the skin. By relocation of the laser active area on screen surface, a large portion of human face could be covered. To achieve that, very specific wavelengths are used, since most of lasers’ wavelengths do not penetrate into the skin, making skin rejuvenation expensive and difficult. It is our purpose to use laser energy from a wide wavelengths’ spectrum, applied in a boring-like procedure, by applying successive focused lasers penetrating into the skin by shifting the focal point in such a way that it penetrates from epidermis to deep dermis. This procedure will produce ablated columns, which will stimulate collagen remodeling while leaving intact areas in between which will lead to a fast epithelial recovery.

### BACKGROUND OF THE INVENTION

### 1. Field of Invention

The present invention relates to an apparatus for drilling miniature columns into the skin for stimulating collagen recovery, ameliorating the total wrinkles on treated person. The drilling function is performed by shifting the focal point of multiple laser beams from epidermis area to a predeterminate depth, while ablating the skin to create penetrating columns.

### 2. Description of the Related Art

The disclosed art is based on a laser drilling device and procedure for skin rejuvenation system, ameliorating wrinkles and skin textures, using focused laser beams over a wide spectrum, by shifting the focal position of said focused beams from epidermis to a predeterminate depth into the patient’s dermis multiple penetrating miniature shafts are created.

For aesthetic treatments, lasers use specific wavelengths to penetrate into the skin. Penetration depth is dictated by light transmission and absorption of skin surface and specific chromophores. Many non-invasive into the skin laser devices are used for treating and improve texture and fine wrinkles, including hair removal by photothermolysis. Ablating and penetrating technologies are also known, especially those using CO2 lasers and Erbium laser resurfacing. Moreover, laser using multiple beams are delivered in a technology called Fraxel, which sends small beams, usually from Erbium lasers, and other IR lasers around 1550 and 1900 nm, to produce fractional injury into the treated person. This procedure based on miniature damaging columns into the skin, stimulates the skin to rejuvenate without causing wide damage epidermis, resulting in fast recovery with impressive results. However, this Fraxeling technology is bounded to use very specific laser wavelengths and have no control over penetration depths which depends on user skin optical characteristics, resulting in a relatively complicated and expensive system. It is the purpose of presented art to offer a solution free of the drawbacks of prior arts using a device which focuses the laser beam and actively moves the focal point, creating very high-density levels at focal point, which are driven into the skin, ablating layer by layer. For best performance, the device may use pulsed laser technology as well.

The device described wherein was designed to create a fractional approach by focusing laser beams to cause dermal shaft-like injury, enabling using multiple wavelengths to create a column by thermal ablation effects, which prevents bleeding and improved skin rejuvenation effect.

### SUMMARY

The present invention offers a solution to main limitation of fractional non ablative dermal injury systems, which are very expensive due to the specific wavelengths used to perform the fractional ablations, and the fact that it’s not a tightly controlled procedure. This leads to an expensive system with modest results because of the technological limitations. The disclosed art offers a solution based on focused laser technology which creates high power densities on skin surface, and it’s driven into the skin by lowering the focal point positioning, successively ablating skin layers as the focal point is further shifted into the skin. This results into a controllable procedure since the user can decide and adjust the penetration depths as well as the size of the focused beam, controlling the laser density.

More particularly, the invention provides an apparatus of boring multiple deep holes into the skin by means of ablation using high density focused beams, traversing downwards and successively ablating skin surfaces. An ideal laser source for this application could be VCSEL, or alternatively multiple laser diodes on a single chip activated in parallel. The creation of micro-column ablation into the skin while leaving major untouched islands will lead to short-term recovery, having an effect of skin rejuvenation.

To summarize, the art discloses a device and method for skin rejuvenation device for treatment of a selected area by multiple focused lasers based on: an optical bench mounted with multiple LD configured to emit the laser beams towards said skin selected area through a focusing optical element configured to slide up and down and focus the beams on skin surface and below through successive positioning moves, a motor system configured to move the optical focusing element onto the skin surface and below to achieve laser boring on multiple points perpendicular to skin surface, a controller system configured to activate the motor system in a successive mode, synchronized with laser pulses and position, and a pulse generator to control power output of the multiple treatment lasers to be applied upon the treatment area and driven to a certain depth relative to epidermis. Said laser source may have wavelengths according to different skin types. Moreover, an external camera could be used to recall the treatment session.

### BRIEF DESCRIPTION OF THE DRAWINGS

Various embodiments are illustrated below. These figures are not drawn to scale and schematically describe the invention, but do not limit its applications.

FIG. 1 is representing the various laser wavelengths used for skin treatment and their preferred applications.

FIG. 2 shows the procedure of boring into the skin as performed by proposed rejuvenating laser drilling apparatus.

FIG. 3 is a schematic assembly drawing of proposed rejuvenating laser drilling apparatus.

### DETAILED DESCRIPTION OF THE DRAWINGS

Human skin has properties that dictate the penetration and absorption of light by skin. For example, specific laser beams are used for penetrating, creating dermatological rejuvenation which have a spectral emission consistent with Er:YAG lasers (2.9 microns) and CO2 lasers (10.6 microns). The said Er:YAG lasers and CO2 lasers are characterized by shallow penetration into the skin but with high level of power. The device described wherein was designed to create a fractional approach to dermal injury, enabling using multiple wavelengths to create a column by thermal ablation effects, which prevents bleeding and improved skin rejuvenation effect. For our application, the most important biological effect will be laser absorption and ablation, which is generated by skin absorbing the laser light and transforming it into heat, to increase the temperature of the tissue exposed to laser radiation. In order to create an ablation effect which is adequate for our application, the laser density should be high to such a level that will generate skin ablation. Although other interactions with skin are common, such as transmission, reflection and scattering are also participating, the most important in disclosed technology is ablation caused by focused laser beam and increased absorption. Since the initial interaction will darken the affected area, the absorption should increase and will facilitate further penetration to various wavelengths that are not common for this application.

The high focused beam creates extremely high-density level to such an extent that even if skin absorption for a specific wavelength is low, the high-density offsets this effect and will penetrate the skin at a shallow depth. Multiple laser activation, each targeted to a different depth, will generate a long shaft into the skin as required by the specific application. This is achieved with relatively low-cost laser diodes, increasing the overall treatment accessibility.

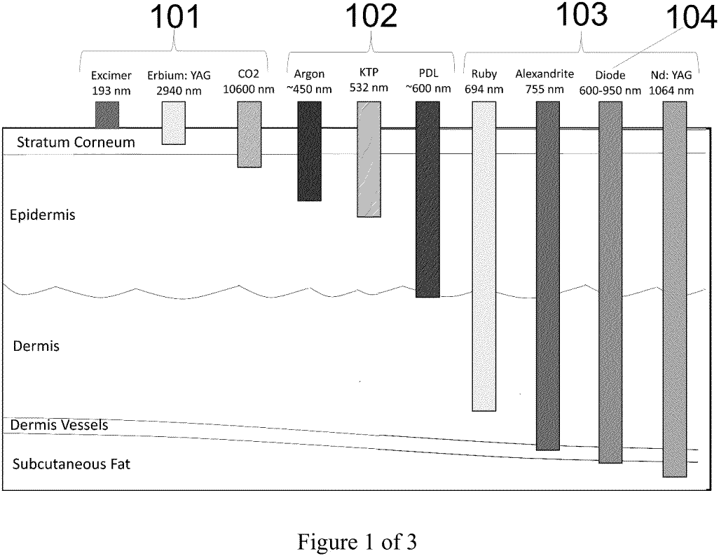

FIG. 1 illustrates the common uses of lasers in prior art and their applications, in accordance with this figure penetrating lasers are usually very expensive, work by creating a shaft into the skin without sequential progress. 101 represents the lasers with low penetration and high power, used for Fraxel penetrating treatment, and are relatively rare and expensive. 102 are lasers primarily used for photodynamic treatment, which are out of the scope of this invention. 103 are common lasers used for non-invasive hair removal and rejuvenation, offering low-cost and proliferation due to recent advancements in laser diodes technologies. By applying the successive penetration concept, our disclosed art will enable the usage of various wavelengths selection, preferable in the near-IR between 600 and 950 nm, denoted as 104. The technology will allow using laser diodes which are in the visible range and are potentially effective for usage with laser boring technology.

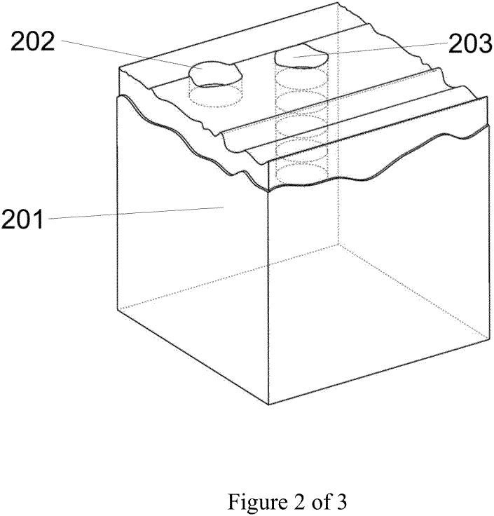

FIG. 2 shows a cross-section of the human skin and displays in an explanatory way the procedure of boring into the skin as performed by proposed rejuvenating laser drilling apparatus. 201 is a 3-D cross section of human skin, to be treated by our laser boring procedure. 202 shows the effect of highly focused beam with high levels of power distribution on initial boring. 203 further shows the removal of sequential layers as the laser beam focus point is lowered into the skin with the final result of a bored skin section. The penetration depth is dictated by the number of successive laser pulses.

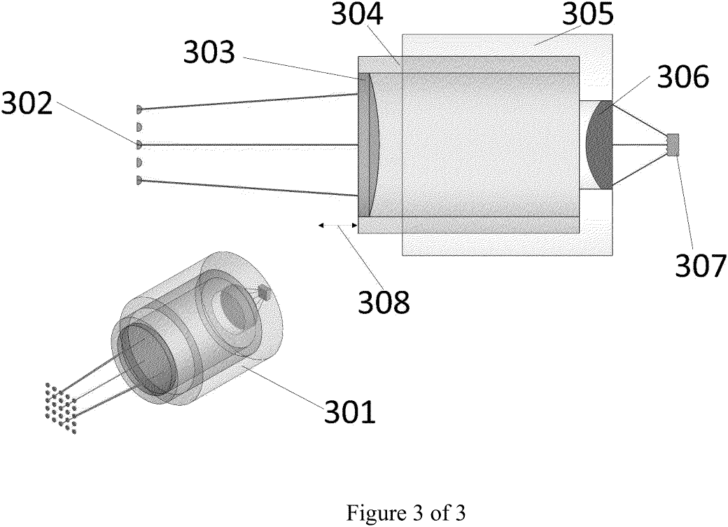

FIG. 3 describes by way of example the proposed optical system which includes a VCSEL array with multiple lasers lasing in parallel, a collecting and collimating optics and the focusing optics which are mounted on a voice coil motor capable of moving up and down the focusing optics as required. The collimating optics are configured in such a way that its image plane coincides with said VCSEL. For example, the said collimating optics has a focal length of f1. The focusing lens will collect the collimated beams produced by the lens with f1 focal length, and focus it at the skin at the appropriate level. If we’ll assign f2 to the second lens, then we can calculate the approximate magnification of VCSEL array at skin level, by the ratio of f2/f1.

The figure has a 3-D reconstruction of the proposed exemplary and non-limiting rejuvenating laser drilling apparatus, and is denoted as 301. A cross section of 301 is further displayed for better understanding the role of each component. 302 represents the magnified laser array emitted by VCSEL. 303 is the focusing optics collected from collimating optics. Said 303 lens is mounted on a voice coil denoted as 304 capable of moving back and forth according to electronic signals. 305 is the magnetic stationary housing which performs as a housing for collimating lens 306. 307 represents the said VCSEL. The back-and-forth possible linear movement of said coil 304 is denoted as 308.

What is claimed is:

1. A skin rejuvenation device for treatment of a selected area by multiple focused lasers comprising:
an optical bench mounted with multiple LD configured to emit the laser beams towards said skin selected area through a focusing optical element configured to slide up and down and focus the beams on skin surface and below through successive positioning moves;
a motor system configured to move the optical focusing element onto the skin surface and below to achieve laser boring on multiple points perpendicular to skin surface;
a controller system configured to activate the motor system in a successive mode, synchronized with laser pulses and position; and
a pulse generator to control power output of the multiple treatment lasers to be applied upon the treatment area and driven to a certain depth relative to epidermis.

2. The skin rejuvenation device according to claim 1, wherein laser sources have different wavelengths according to different skin types.

3. The skin rejuvenation device according to claim 1, wherein a mounted camera is configured to record treatment session.

4. The skin rejuvenation device according to claim 1, wherein said laser sources are based on VCSEL array.

5. A method for a dermatological skin rejuvenation device, treating selected skin area by multiple boring focused lasers comprising:
an optical bench mounted with multiple LD configured to emit the laser beams towards said skin selected area through a focusing optical element configured to slide up and down and focus the beams on skin surface and below through successive positioning moves;
a motor system configured to move the optical focusing element onto the skin surface and below to achieve laser boring on multiple points perpendicular to skin surface;
a controller system configured to activate the motor system in a successive mode, synchronized with laser pulses and position; and
a pulse generator to control power output of the multiple treatment lasers to be applied upon the treatment area and driven to a certain depth relative to epidermis.
